(19) United States
(12) Patent Application Publication
(10) Pub. No.: US 2021/0000480 A1
(43) Pub. Date: Jan. 7, 2021

(54) EMBOLUS MATERIAL

(71) Applicant: TERUMO KABUSHIKI KAISHA, Tokyo (JP)

(72) Inventors: Eri IKUNO, Ashigarakami-gun (JP); Yuki ITOH, Hadano-shi (JP); Yohichi SAKAGAWA, Isehara-shi (JP)

(73) Assignee: TERUMO KABUSHIKI KAISHA, Tokyo (JP)

(21) Appl. No.: 17/028,114

(22) Filed: Sep. 22, 2020

Related U.S. Application Data
(63) Continuation of application No. PCT/JP2019/011513, filed on Mar. 19, 2019.

(30) Foreign Application Priority Data
Mar. 22, 2018 (JP) 2018-054613

(51) Int. Cl. A61B 17/12

(52) U.S. Cl. CPC A61B 17/12113; A61B 2017/00867; A61B 2017/1205; A61B 17/1214

(57) ABSTRACT

An embolus material for inhibiting the inflow of blood into an aortic aneurysm from a branched blood vessel by filling, via a catheter, the inside of the aortic aneurysm joined to the branched blood vessel includes: an elongated filling body that is formed to be narrower than the inside diameter of the catheter; and a shaping section that is provided at a portion of the filling body and that forms a structure larger than the width of the branched blood vessel after exiting from the catheter.

### CROSS-REFERENCES TO RELATED APPLICATIONS

This application is a continuation of International Application No. PCT/JP2019/011513 filed on Mar. 19, 2019 which claims priority to Japanese Patent Application No. 2018-054613 filed on Mar. 22, 2018, the entire content of both of which is incorporated herein by reference.

### FIELD OF THE INVENTION

The present invention generally relates to an embolus material used to treat an aneurysm formed in a blood vessel.

### BACKGROUND DISCUSSION

A method of providing an artificial blood vessel through laparotomy or thoracotomy has been used to treat an aortic aneurysm, but this method is a rather invasive treatment and imposes a great burden on a patient. Thus, in recent years, transition to stent graft treatment of performing treatment in a blood vessel has occurred.

The stent graft treatment is a treatment that inhibits blood from flowing into the aortic aneurysm by inserting a catheter from the patient's femoral artery and providing an artificial blood vessel (stent graft) with a spring in a target lesion.

However, it is known that the stent graft treatment causes several complications. One of the complications is Type 2 endoleak. Type 2 endoleak is a complication in which a blood flow that was flowing out in a forward direction from a branch blood vessel located in the aortic aneurysm part to a terminal blood vessel is reversed from the terminal blood vessel side to the inside of the aneurysm due to the provision of a stent graft, and thus the blood flow remains in the aneurysm. If this is left unattended for a long period of time, the aortic aneurysm may be enlarged by the reversed blood flow. Such Type 2 endoleak is said to occur in about 20% to 30% of patients who have undergone stent graft treatment in the long term.

As a method for treating Type 2 endoleak, a method has been proposed in which the origin of a branch vessel that is branched from an aortic aneurysm is occluded with an embolus coil. This method is called “Coil embolization”. A method of filling a liquid embolus material into an aortic aneurysm has been proposed in “A Novel and Simple Technique for Embolization of Type 2 Endoleaks Through Direct Sac Access From the Distal Stent-graft Landing Zone”, G. Coppi et-al., European Journal of Vascular and Endovascular Surgery, Volume 47 Issue 4 p. 394 to 401, April/2014. This method is called “Sac embolization”. Japanese Patent Application Publication No. 202-539853 (JP-T-2002-539853) and Japanese Patent Application Publication No. 202-537353 (JP-T-2004-537353) disclose a method of filling a gelatinous embolus material.

### SUMMARY

However, there may be a plurality of branch vessels connected to an aortic aneurysm. Since some branch blood vessels are thin or complicated, it is hard to access all branch blood vessels, and it is difficult to occlude all branch blood vessels with an embolus coil. Currently, treatment of occluding a main branch blood vessel connected to an aortic aneurysm is performed, but there is a problem in that inflow of blood from a branch blood vessel that cannot be treated is not prevented, and the recurrence rate is high.

In the treatment method using a liquid embolus material, a liquid embolus material that is solidified when contacted with blood is injected into an aortic aneurysm, and thus all blood vessels connected to the aortic aneurysm are blocked.

However, when the liquid embolus material is injected into the aortic aneurysm, the liquid embolus material before being solidified flows out to a peripheral portion of a branch blood vessel and distally embolizes a blood vessel at a part separated from the aortic aneurysm, and thus there is a problem in that a new complication is caused.

Therefore, as a method of blocking all blood vessels connected to an aortic aneurysm without accessing all branch blood vessels, it is considered that a long embolus material that is fillable via a catheter is filled into the aortic aneurysm, but an embolus material that is fillable via a catheter may be thinner than some branch blood vessels connected to the aortic aneurysm. In this case, the embolus material flows out to a peripheral portion of a branch blood vessel in the same manner as a liquid embolus material, and thus there is concern called distal embolization that a blood vessel at a part separated from the aortic aneurysm is distally embolized.

The embolus material disclosed here is capable of inhibiting blood from flowing into an aortic aneurysm while preventing the occurrence of a complication due to the entire embolus material flowing out of a branch blood vessel connected to the aortic aneurysm.

An embolus material according to one aspect includes an elongated filling body that is sized smaller than an inner diameter of the catheter so that the elongated filling body is positionable in the catheter; and a shaped portion provided at least at a part of the elongated filling body. The shaped portion is configured to bend in a bent state or curve in a curved state after coming out of the catheter. The shaped portion is configured to spread out, in the bent or curved state, wider than an inner diameter of the branch blood vessel after coming out of the catheter.

When an aneurysm is accessed from a branch blood vessel, or when the aneurysm is accessed through transcealing, a thickness of a catheter may be restricted due to difficulty in the access, and thus a small embolus material may have to be injected from a catheter smaller than an inner diameter of a branch blood vessel. Even in such a case, according to the embolus material described above, since the shaped portion having a width larger than that of the branch blood vessel is provided in a part of the embolus material, it does not occur that the entire embolus material strays into the branch blood vessel and thus occludes an unintended location. Consequently, it is possible to prevent a reverse flow of blood into an aneurysm without causing a new complication. In a case where an embolus material smaller than an inner diameter of a branch blood vessel, an effect that a portion other than the shaped portion enters the branch blood vessel and thus directly embolizes the branch blood vessel can be expected.

In the embolus material, the filling body may be made of a hydrogel that is swollen through contact with blood. Here, the hydrogel is a polymer material that is swollen through contact with water or a solvent and thus increases in volume. The hydrogel also includes a stimulus-responsive gel material of which a volume increases in response to stimuli such as pH, a temperature, water, and an ionic strength. Such an embolus material does not adhere to a tip of a catheter unlike a liquid embolus material, and thus can be easily handled by using the catheter. The filling body is swollen after being provided, and thus an amount of the provided embolus material is small. The embolus material does not entirely bury a blood vessel aneurysm immediately after being provided, gradually buries the aneurysm by being swollen, and is thus excellent in safety without causing a sudden pressure increase.

In the embolus material, the shaped portion may include a shape memory member wound in a coil shape on an outer circumferential portion of the filling body. The shape memory member is a member deformed into a predetermined shape due to a temperature, a solvent (water), pH, an ionic strength, or the like. Such a shape memory member is shaped into a predetermined shape in advance, and thus a terminal end of a filling body discharged from a catheter can be deformed to form a structure larger than a branch blood vessel. As a result, it is possible to prevent an embolus material from flowing into a branch blood vessel.

In the embolus material, the shaped portion may include a shape memory member buried or embedded in the filling body. It is possible to form a structure larger than an inner diameter of a branch blood vessel at a terminal portion of the filling body by using the shape memory member. Consequently, it is possible to prevent the embolus material from flowing into a branch blood vessel.

In the embolus material, the shaped portion may be shaped through polymerization in a mold having a shape of the structure. Since a crosslinking structure of molecules forming the filling body reflects a shape in the mold at the time of synthesis, a pre-shaped shape is shown when the provided filling body is swollen. Consequently, it is possible to prevent an embolus material from flowing into a branch blood vessel.

In the embolus material, the shaped portion may be formed of an elastic member or a shape memory member coupled to a terminal end of the filling body. Consequently, it is possible to prevent an embolus material from flowing into a branch blood vessel.

In the embolus material, the shaped portion may be formed at both ends of the filling body. The shaped portion may be formed over the entire filling body. It is also possible to prevent an embolus material from flowing into a branch blood vessel by using the configurations. Since the terminal end of the embolus material is shaped, the risk of the distal end thereof sticking to an aneurysm portion and puncturing the aneurysm portion can be reduced.

In the embolus material, the shaped portion may be shaped so as to spread out in a range wider than the inner diameter of the branch blood vessel in a width direction of the filling body by bending with respect to the filling body by bending with respect to the filling body. Consequently, it is possible to prevent an embolus material from flowing into a branch blood vessel.

In the embolus material, the shaped portion may be shaped into a circular or polygonal ring shape having a diameter larger than the inner diameter of the branch blood vessel. The shaped portion may be shaped into an umbrella shape having a diameter larger than the inner diameter of the branch blood vessel by a terminal end of the branched filling body being rewound to a proximal side of the filling body. The shaped portion may be shaped such that a terminal end of the branched filling body forms a ball-like shape having a diameter larger than the inner diameter of the branch blood vessel. Such a shaped portion can also prevent an embolus material from entering a branch blood vessel.

The embolus material is used to treat an aortic aneurysm, and the shaped portion may be configured to forma structure having a diameter of 3 mm or more. Consequently, since a width of the structure of the shaped portion is larger than an inner diameter of a branch blood vessel connected to an aortic aneurysm, for example, the inferior mesenteric artery having a thickness of about 3 mm or the lumbar artery having a thickness of about 2 mm, it is possible to prevent the embolus material from entering the branch blood vessel. Such an embolus material is suitable for treating Type 2 endoleaks.

In the embolus material, the filling body may have a diameter smaller than the inner diameter of the branch blood vessel in a state before being swollen such that a part of the filling body other than the shaped portion may enter and occlude the branch blood vessel. Consequently, the embolus material is filled into an aneurysm, and an effect that a portion other than the shaped portion enters a branch blood vessel and thus directly embolizes the branch blood vessel can also be expected.

In the embolus material, a linear portion not being shaped may be disposed at a terminal portion of the filling body. According to the embolus material, the linear portion can easily enter a branch blood vessel, and thus an effect that the linear portion having entered the branch blood vessel occludes the branch blood vessel can be expected.

The embolus material can inhibit blood from flowing into an aortic aneurysm while preventing the occurrence of a complication due to the entire embolus material flowing out of a branch blood vessel connected to the aortic aneurysm.

According to another aspect, a method comprises: introducing a catheter into a body while an embolus material is positioned inside the catheter, with the embolus material comprising an elongated filling body and a shaped portion provided at least at a part of the elongated filling body; and pushing the embolus material through an open distal end of the catheter while the catheter is in the body so that the embolus material comes out of the catheter and is introduced into an aortic aneurysm to inhibit inflow of blood into the aortic aneurysm from a branch blood vessel or outflow of the blood from an inside of the aortic aneurysm to the branch blood vessel. The shaped portion of the embolus material bends or curves after coming out of the catheter to spread out in a bent or curved state wider than an inner diameter of the branch blood vessel.

### BRIEF DESCRIPTION OF THE DRAWINGS

FIG. 1 is a perspective view illustrating an embolus material according to a first embodiment.

FIG. 2 is a schematic configuration diagram illustrating an example of using the embolus material in FIG. 1 for an aortic aneurysm.

FIG. 3 is a schematic configuration diagram illustrating an example of an operation of the embolus material in FIG. 1.

FIG. 4 is a perspective view illustrating an embolus material according to a second embodiment.

FIG. 5A is a plan view illustrating an embolus material according to a third embodiment, FIG. 5B is a cross-sectional view taken along the line VB-VB in FIG. 5A, and FIG. 5C is a cross-sectional view taken along the line VC-VC in FIG. 5A.

FIG. 6 is a perspective view illustrating an embolus material according to a fourth embodiment.

FIG. 7A is a plan view illustrating a part of a mold used to manufacture the embolus material in FIG. 6, FIG. 7B is a plan view illustrating a state in which a raw material for the embolus material flows into the mold in FIG. 7A, and FIG. 7C is a plan view illustrating the embolus material formed by the mold in FIG. 7A.

FIG. 8 is a perspective view illustrating a terminal portion of an embolus material according to a fifth embodiment.

FIG. 9 is a perspective view illustrating a terminal portion of an embolus material according to a sixth embodiment.

FIG. 10 is a side view illustrating a terminal portion of an embolus material according to a seventh embodiment.

FIG. 11A is a perspective view illustrating a first configuration example of a shaped portion in FIG. 10, FIG. 11B is a perspective view illustrating a second configuration example of the shaped portion in FIG. 10, FIG. 11C is a perspective view illustrating a third configuration example of the shaped portion in FIG. 10, and FIG. 11D is a perspective view illustrating a fourth configuration example of the shaped portion in FIG. 10.

FIG. 12A is a perspective view illustrating a fifth configuration example of the shaped portion in FIG. 10, and FIG. 12B is a perspective view illustrating a sixth configuration example of the shaped portion in FIG. 10.

FIG. 13 is a side view illustrating a seventh configuration example of the shaped portion in FIG. 10.

FIG. 14A is a perspective view illustrating a shaped portion of an embolus material according to an eighth embodiment, and FIG. 14B is a perspective view illustrating a modification example of the shaped portion in FIG. 14A.

FIG. 15 is a perspective view illustrating a shaped portion of an embolus material according to a ninth embodiment.

FIG. 16 is a perspective view illustrating a shaped portion of an embolus material according to a tenth embodiment.

FIG. 17 is a perspective view illustrating an embolus material according to an eleventh embodiment.

FIG. 18 is a perspective view illustrating a shaped portion of an embolus material according to a twelfth embodiment.

### DETAILED DESCRIPTION

Set forth below with reference to the accompanying drawings is a detailed description of embodiments of an embolus material representing examples of the inventive embolus material disclosed here.

### First Embodiment

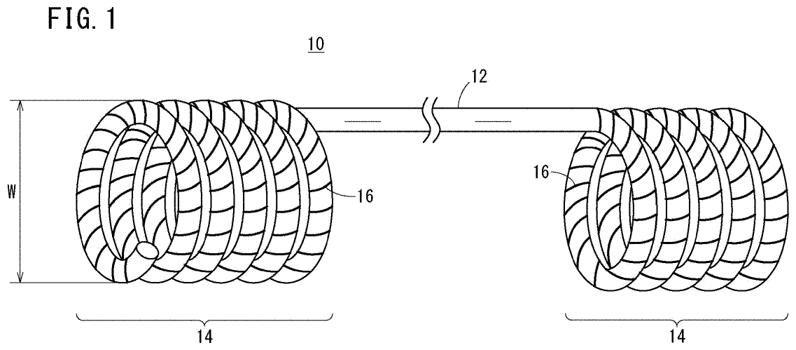

As illustrated in FIG. 1, an embolus material 10 according to the present embodiment disclosed as an example of the inventive embolus material includes an elongated filling body 12 forming an embolus material main body, and shaped portions 14 which may be provided at both ends of the filling body 12. The embolus material 10 is provided in an aneurysm such as an aortic aneurysm via a catheter, and the shaped portions 14 form coil-shaped structures. Hereinafter, details of the embolus material 10 will be described.

The filling body 12 is formed in a solid thread-like or elongated shape, and the entire length of the filling body 12 is, for example, about 10 cm to 100 cm. A diameter of the filling body 12 is slightly smaller than an inner diameter of a catheter used to access an aortic aneurysm. The outer diameter of the filling body 12 may be, for example, about 1 mm. The filling body 12 is not limited to a solid thread-like or elongated shape, and may be formed in a hollow tubular shape. A cross-sectional shape of the filling body 12 is not limited to a circular shape, and may be an elliptical shape, a triangular shape, a rectangular shape, or a polygonal shape, and a combined shape thereof.

The filling body 12 is made of a polymer material that is swollen by water in blood, and is configured to be swollen after being provided in an aortic aneurysm and thus to be filled into the aortic aneurysm. As such a polymer material, a hydrogel may be used. The hydrogel is manufactured by reacting a mixture containing (a) a monomer or polymer at least a part of which is sensitive to environmental changes such as a pH value, a temperature, water, and an ionic strength, (b) a crosslinking agent, and (c) a polymerization initiator.

As (a) the monomer or polymer, for example, ethylenically unsaturated monomers (for example, acrylic acid-based monomers or amine-based monomers, N,N′-methylenebisacrylamide, N-vinylpyrrolidinone, 2-hydroxyethyl methacrylate, and N-isopropylacrylamide), monomers containing polyols or polyglycols, and derivatives thereof may be used. In addition, a hydrogel composed of a polysaccharide polymer may be used.

The filling body 12 is swollen through contact with blood by virtue of the composition, and is expanded to a size of 2 to 4 times in a radial direction. A diameter of the filling body 12 after being expanded is about 2 mm to 4 mm. Since this diameter is equal to or greater than about 2 mm corresponding to an inner diameter of the lumbar artery that is a typical branch blood vessel connected to an aortic aneurysm, or about 3 mm corresponding to an inner diameter of the inferior mesenteric artery, there is little concern that the filling body 12 after being expanded will stray into the branch blood vessel. However, since the filling body 12 before being expanded has a diameter of about 1 mm and is thus small, there is concern that the filling body before being expanded may stray into a large branch blood vessel.

Therefore, the embolus material 10 of the present embodiment is provided with the shaped portions 14 forming structures each having a diameter larger than an inner diameter of a branch blood vessel at terminal portions of the filling body 12. When positioned in a catheter, each shaped portion 14 forms a structure that has a linear shape in the same manner as other portions of the filling body 12 in a catheter, but the shaped portion 14 is deformed into a predetermined shape after being discharged from the catheter, and thus has a size not able to enter a branch blood vessel, at the terminal portion of the filling body 12.

In the present embodiment, each shaped portion 14 is shaped such that the terminal portion of the filling body 12 forms a structure curved in a coil shape. A diameter W of the coil-shaped portion is more than about 2 mm to 3 mm corresponding to an inner diameter of a branch blood vessel. That is, the inner diameter of a branch vessel is typically about 2 mm to 3 mm, and the diameter W is more than that inner diameter. Therefore, in the filling body 12 used to treat an aortic aneurysm, the diameter W is preferably 3 mm or more. In a case where a curvature radius of the curve of the structure is too large, the terminal portion of the filling body 12 easily enters a branch blood vessel in the same manner as the linear filling body 12, and thus the curvature radius of the curve is preferably equal to or less than twenty times the inner diameter of the branch blood vessel.

The shaped portion 14 of the present embodiment is formed by winding a thin wire 16 formed of a shape memory member in a coil shape on an outer circumference of the filling body 12. A diameter (wire diameter) of the thin wire 16 may be about 0.01 to 0.5 mm, and may employ a wire thinner than the filling body 12.

Examples of the shape memory member or shape memory material from which the thin wire 16 may be fabricated include a shape memory alloy or a shape memory resin. Examples of the shape memory alloy include Ni—Ti-based alloys (Nitinol), Cu—Al—Ni-based alloys, Cu—Sn-based alloys, Cu—Zn-based alloys, Cu—Zn—X (X═Si, Al, or Sn)-based alloys, Fe—Pt-based alloys, Mn—Cu-based alloys, Fe—Mn—Si-based alloys, Fe—Ni—Co—Al-based alloys, Pt-based shape memory alloys, Co—Ni—Al-based alloys, Co—Ni—Ga-based alloys, Ni—Fe—Ga-based alloys, and Ti—Pd-based alloys.

Regarding the shaped end portions 14, the terminal portions of the filling body 12 on which the thin wires 16 are wound are shaped in advance into the illustrated coil shape, and then the shaped portions 14 are provided in a state in which each shaped portion is linearly stretched. The embolus material 10 coming out of a catheter forms the structure illustrated in FIG. 1 as a result of the thin wires 16 of the shaped portions 14 being deformed into the pre-shaped coil shape at a body temperature. FIG. 1 shows that the embolus material may include a shaped portion 14 at each end, though this embodiment of the embolus material (and the other embodiments described below) may include a shaped portion at only one end.

The embolus material 10 of the present embodiment has the above-described configuration, and, hereinafter, an operation or use of the embolus material 10 will be described.

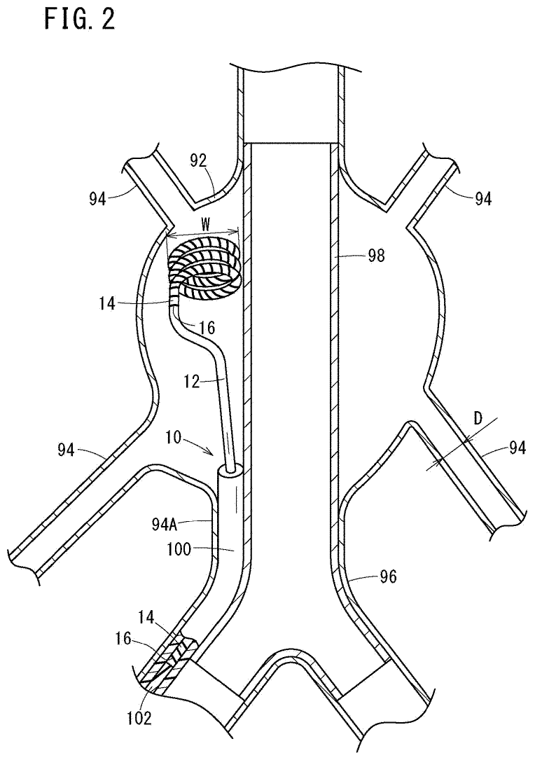

As illustrated in FIG. 2, the vicinities of normal blood vessels 94A located above and below an aortic aneurysm 92 are blocked by a stent graft 98 provided inside a blood vessel 96. The aortic aneurysm 92 is connected to a plurality of branch blood vessels 94 toward terminal portions such as the leg, the bowel, and the spinal cord. The embolus material 10 can be introduced into the aortic aneurysm 92 via a catheter 100 provided between the stent graft 98 and the blood vessel 96. The catheter 100 may be provided in advance between the stent graft 98 and the blood vessel 96 when the stent graft 98 is provided.

For example, as disclosed in Japanese Patent Application Publication No. 2002-539853 (JP-T-2002-539853), after the stent graft 98 is provided, a method may be employed in which the catheter 100 is inserted while making a gap between the stent graft 98 and the blood vessel 96, to secure access to the aortic aneurysm 92. There may be a method of accessing the aortic aneurysm 92 by causing the catheter 100 to pass through the branch blood vessel 94 connected to the aortic aneurysm 92. There may also be a method of directly inserting the catheter 100 into the aortic aneurysm 92 by directly puncturing the aortic aneurysm 92.

The thread-like or elongated embolus material 10 having the terminal portions shaped in advance into a predetermined shape is inserted into the catheter 100 that is inserted into or positioned in the aortic aneurysm 92. The embolus material 10 may be pushed out by a pushout member 102 inserted from a proximal portion side of the catheter 100, and the embolus material 10 is located in the aortic aneurysm 92 from a distal end of the catheter 100. Thereafter, the catheter 100 is extracted from the aortic aneurysm 92 and the blood vessel 96.

In the embolus material 10 positioned in the aortic aneurysm 92, the thin wire 16 made of a shape memory alloy wound on the terminal portion of the filling body 12 is deformed into a pre-shaped coil shape due to a body temperature. That is, the temperature existing in the living body causes the wire(s) 16, and thus the terminal portion(s) 14 of the filling body 12 positioned in the aortic aneurysm 92, to be deformed so that the terminal portion(s) 14 exhibits a coil shape such as illustrated in FIG. 2. As a result, a structure with a width W greater than an inner diameter D of the branch blood vessel 94 is formed at the terminal portion of the embolus material 10. Consequently, the embolus material 10 is prevented from flowing into the branch blood vessel 94. The filling body 12 of the embolus material 10 is gradually swollen by water in blood, to fill the inside of the aortic aneurysm 92. The embolus material 10 may be provided a plurality of times until the swollen filling body 12 is filled into the inside of the aortic aneurysm 92. That is, a plurality of the embolus material 10 may be positioned in the aortic aneurysm 92.

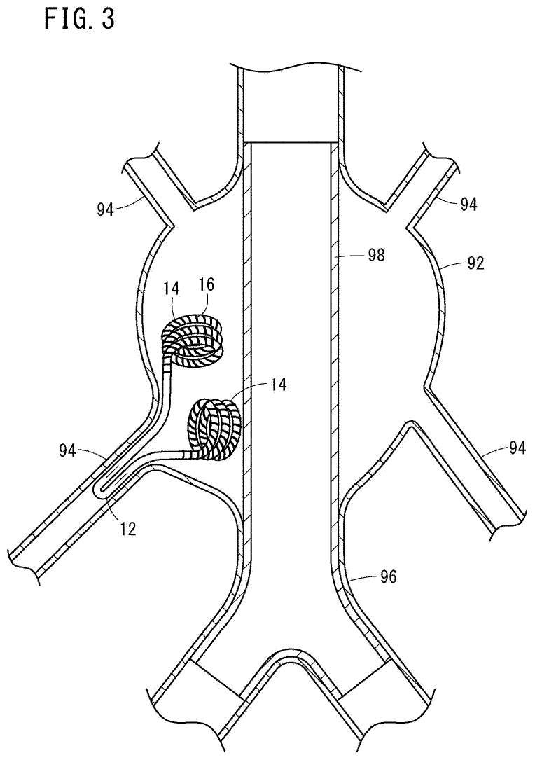

The present inventors have made tests and discovered that a portion other than the shaped portion 14 may enter the branch blood vessel 94 and thus partially embolizes the branch blood vessel 94. In other words, as illustrated in FIG. 3, it is clear to achieve an effect that the linear portion of the filling body 12 may enter the branch blood vessel 94 in a bent state, to be then swollen in the branch blood vessel 94, and thus directly embolizes the branch blood vessel 94. Even in this case, since the shaped portions 14 are formed in the embolus material 10, the entire embolus material 10 does not distally flow out to the branch blood vessel 94, and there is little concern of causing a complication. As mentioned above, according to the embolus material 10 of the present embodiment, it can be expected to achieve an effect that the embolus material fills the aortic aneurysm 92 and also directly embolizes the branch blood vessel 94.

As mentioned above, according to the embolus material 10 of the present embodiment, the embolus material can be shaped to spread out (increase in size) in a range wider than the inner diameter D of the branch blood vessel 94 in the width direction of the filling body 12. The shaped portion 14 can prevent the entire embolus material 10 from entering the branch blood vessel 94. As a result, the inside of the aortic aneurysm 92 or the branch blood vessel 94 can be filled with the embolus material 10 and so it is possible to inhibit blood from flowing into the aortic aneurysm 92 while preventing the occurrence of a complication caused by embolizing a blood vessel at a part separated from the aortic aneurysm 92.

The filling body 12 forming the embolus material 10 is made of a hydrogel that is swollen through contact with blood, and thus an amount of the provided embolus material 10 may be small. In this case, since the embolus material is not filled into the aortic aneurysm 92 or the branch blood vessel 94 immediately after being provided but is swollen to be gradually buried into the aortic aneurysm 92 or the branch blood vessel 94, the embolus material does not cause a sudden pressure increase in the aortic aneurysm 92 or the branch blood vessel 94 and can thus be provided safely.

The filling body 12 of the embolus material 10 is made of a flexible thread-like or elongated gel material, and can thus follow the aortic aneurysm 92 or the branch blood vessel 94 even in a case where a shape thereof is deformed. The filling body 12 of the embolus material 10 before being swollen does not adhere to the tip of the catheter 100 in the same manner as a liquid embolus material, and thus the catheter 100 can be easily handled and be easily provided.

Since the terminal end of the embolus material 10 is shaped, the risk of the distal end of the embolus material 10 sticking to the aortic aneurysm 92 and puncturing the aortic aneurysm can be reduced.

### Second Embodiment

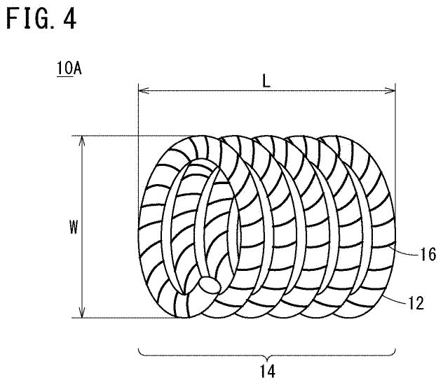

In an embolus material 10A according to another embodiment representing another example of the inventive embolus material 10A disclosed here, as illustrated in FIG. 4A, the entire filling body 12 is formed in a coil shape. In other words, the shaped portion 14 is provided over the entire filling body 12.

The filling body 12 is made of a thread-like or elongated hydrogel in the same manner as the filling body 12 described with reference to FIG. 1, and the thin wire 16 formed of a shape memory member or a shape memory material is wound on an outer circumferential portion of the filling body 12. In the present embodiment, the thin wire 16 is wound from one end of the filling body 12 to the other end of the filling body 12. The thin wire 16 is shaped in advance such that the filling body 12 is deformed into a coil shape as illustrated when coming into contact with a body temperature. The filling body 12 on which the thin wire 16 is wound is provided in a linearly stretched or straightened form.

The width W and the entire length L of a coil-shaped structure formed by the shaped portion 14 are preferably greater than the inner diameter D of the branch blood vessel 94. In a case where the embolus material 10A is used in the aortic aneurysm 92, the inner diameter D of the typical branch blood vessel 94 connected to the aortic aneurysm 92 is about 2 to 3 mm, and thus the width W and the entire length L of the shaped portion 14 before being swollen may be equal to or more than, for example, 3 mm.

The embolus material 10A of the present embodiment is inserted into the catheter 100 (refer to FIG. 2) in a state in which the filling body 12 is linearly stretched (straightened), and is provided or positioned in the aortic aneurysm 92 via the catheter 100. The embolus material 10A after being provided is deformed into the coil shape having the diameter W and the entire length L more than the inner diameter D of the branch blood vessel 94 as illustrated in FIG. 4. Consequently, it is possible to prevent the entire embolus material 10A from straying into the branch blood vessel 94. Since the embolus material 10A is shaped into the coil shape, the risk of the distal end of the embolus material 10A sticking to the aortic aneurysm 92 and puncturing the aortic aneurysm can be reduced.

### Third Embodiment

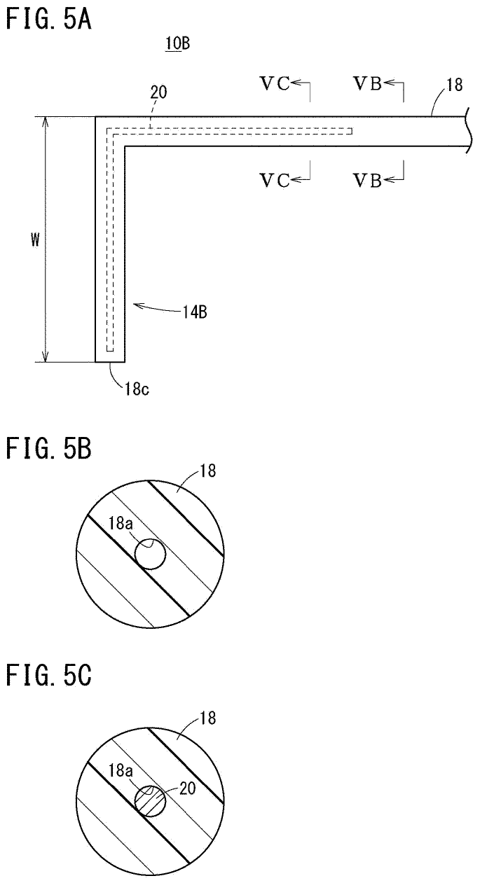

An embolus material 10B according to the present embodiment has a filling body 18 and a shaped portion 14B bent in an L shape at a terminal portion thereof as illustrated in FIG. 5A. FIG. 5A illustrates one end of the embolus material 10B, it being understood that the opposite end of the embolus material 10B may be similarly configured. As illustrated in FIG. 5B, the filling body 18 is provided with a hollow portion 18a. The material from which the filling body 18 is fabricated may be the same material as the material used to make the filling body 12 of the embolus material 10 in FIG. 1.

A core material 20 formed of a shape memory member or a shape memory material is positioned in the hollow portion 18a in the vicinity of a terminal end 18c of the filling body 18 as illustrated in FIG. 5C. The core material 20 has an outer diameter equivalent to an inner diameter of the hollow portion 18a. The core material 20 is made of a shape memory alloy, and is shaped in advance to be bent into an L shape as illustrated when coming into contact with a body temperature.

A shape into which the core material 20 is shaped is not limited to an L shape, and may be a coil shape as illustrated in FIG. 1. The core material 20 may be positioned not only in the vicinity of the terminal portion of the filling body 18 but also along the entire filling body 18 in a total length direction.

The embolus material 10B may be manufactured by shaping the pre-shaped core material 20 into a linear shape at a temperature lower than a body temperature and inserting the core material 20 into the hollow portion 18a from the terminal end 18c of the filling body 18.

When the embolus material 10B is provided in the aortic aneurysm 92 via the catheter 100 (refer to FIG. 2), the core material 20 is deformed into an L shape due to the temperature inside the body. Since the shaped portion 14B deformed into the L shape spreads out in a range W wider than or greater than the inner diameter D of the branch blood vessel 94 connected to the aortic aneurysm 92, the shaped portion 14B is caught at the entrance of the branch blood vessel 94, and thus it is possible to prevent the entire embolus material 10B from straying into the branch blood vessel 94. As mentioned above, the embolus material 10B can be filled into the aortic aneurysm 92 or the branch blood vessel 94 without the entire embolus material straying into the branch blood vessel 94. Since the terminal end 18c is shaped, the risk of the distal end of the embolus material 10B sticking to the aortic aneurysm 92 and puncturing the aortic aneurysm can be reduced.

In the embolus material 10B of the present embodiment, the filling body 18 is not limited to having the hollow portion 18a, and may be formed in a solid thread-like or elongated shape. In this case, the embolus material may be manufactured by placing the linearly stretched core material 20 into a mold after the embolus material is shaped in advance into a predetermined shape and pouring a raw material solution forming a hydrogel around the core material to cause a polymerization reaction to occur such that the core material 20 is buried with the raw material solution.

### Fourth Embodiment

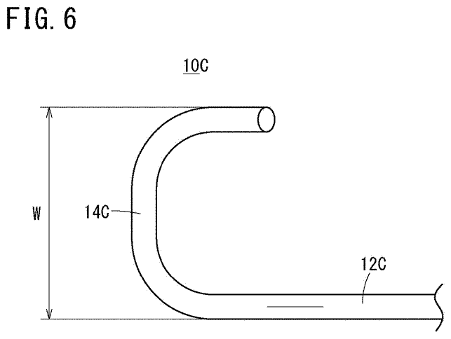

An embolus material 10C according to the present embodiment has a shaped portion (terminal portion) 14C formed by shaping a filling body 12C into a predetermined shape as illustrated in FIG. 6. The shaped portion 14C is formed to spread out or have a dimension in the range W which is greater than the inner diameter D of the branch blood vessel 94. An example in which the illustrated shaped portion 14C is shaped into a C shape is illustrated, but there is no particular limitation thereto, and the shaped portion may be shaped into an L shape, a corrugated shape, or a coil shape. FIG. 6 illustrates one terminal end of the embolus material 10C, it being understood that the opposite terminal end of the embolus material 10C may be similarly configured.

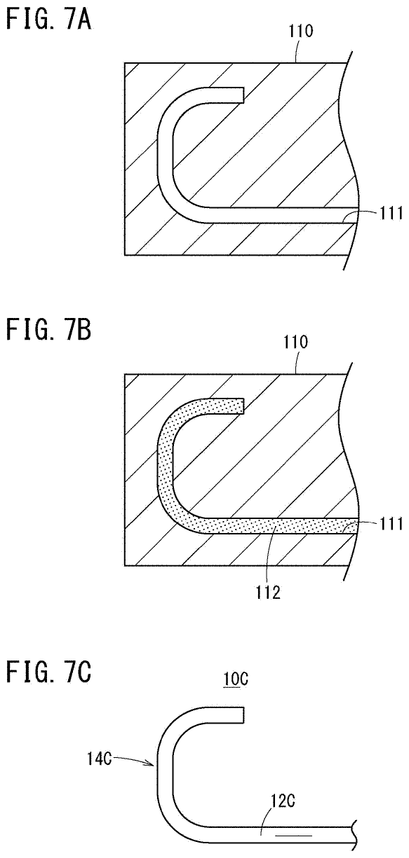

The embolus material 10C of the present embodiment is manufactured according to steps illustrated in FIGS. 7A to 7C. First, as illustrated in FIG. 7A, a mold 110 including a cavity 111 having a shape of the shaped portion 14C at a terminal end thereof is prepared.

Next, as illustrated in FIG. 7B, a raw material 112 for a hydrogel forming the filling body 12C is poured into the cavity 111 of the mold 110. In other words, a mixture containing (a) a hydrophilic monomer or polymer, (b) a crosslinking agent, and (c) a polymerization initiator is poured, and a synthetic reaction is performed in the cavity 111 to form the filling body 12C.

Thereafter, as illustrated in FIG. 7C, the filling body 12C synthesized in the cavity 111 is removed from the mold 110, and thus the embolus material 10C of the present embodiment is obtained. In this embolus material 10C, the shaped portion 14C has flexibility and can be stretched to be made linear by applying force thereto. However, since a crosslinking structure of molecules forming the filling body 12C reflects the shape of the shaped portion 14C at the time of synthesis, the filling body naturally returns to the shape in FIG. 7C when swollen.

The embolus material 10C is provided in a state of being dried and linearly stretched. In a case where the embolus material 10C is provided in the aortic aneurysm 92 via the catheter 100, the embolus material naturally returns to an original shape of the shaped portion 14C when swollen. The embolus material 10C can be provided in the aortic aneurysm 92 while preventing the embolus material from entering the branch blood vessel 94.

As mentioned above, the embolus material 10C of the present embodiment can achieve the same effect as that achieved by the embolus material 10 of the first embodiment.

### Fifth Embodiment

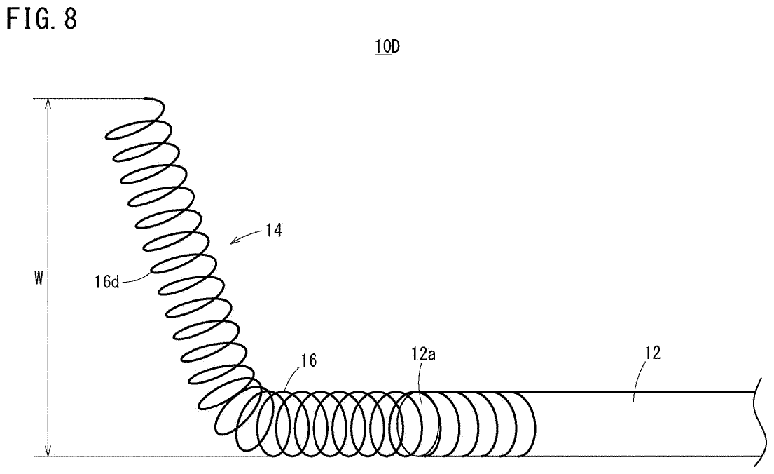

As illustrated in FIG. 8, in an embolus material 10D according to the present embodiment, the thin wire 16 that is shaped into a predetermined shape and is formed of a shape memory member is coupled to the vicinity of an end portion 12a of the filling body 12.

The thin wire 16 is formed in a coil shape, and is coupled to the filling body 12 as a result of a proximal portion of the thin wire 16 being wound on an outer circumferential portion of the elongated filling body 12 around the end portion 12a of the elongated filling body 12. Thus, as shown in FIG. 8, a portion of the thin wire 16 extends beyond the end of the elongated filling body 12. An extending portion 16d of the coil-shaped thin wire 16 on the distal side thereof extends from the end portion 12a of the filling body 12 and is shaped in advance to be bent into an L shape as illustrated when coming into contact with a body temperature. The portion bent into the L shape forms the shaped portion 14. Also in the present embodiment, the shaped portion 14 is formed to spread out in the range W wider or greater than the inner diameter D of the branch blood vessel 94. FIG. 8 illustrates one end of the embolus material 10D, it being understood that the opposite end of the embolus material 10D is similarly configured.

The coil-shaped thin wire 16 of the embolus material 10D is shaped into a linear shape (i.e., without the L-shape) along an axis of the filling body 12 at a temperature lower than the body temperature of the living body and is provided. Consequently, the embolus material 10D can be smoothly inserted into the catheter 100. When the embolus material 10D is provided in the aortic aneurysm 92 via the catheter 100 (refer to FIG. 2), the embolus material 10D forms a structure in which the extending portion 16d of the thin wire 16 is bent in an L shape due to the body temperature, and so it is possible to prevent the embolus material 10D from entering the branch blood vessel 94.

As mentioned above, the embolus material 10D of the present embodiment can achieve the same effect as that achieved by the embolus material 10 of the first embodiment.

### Sixth Embodiment

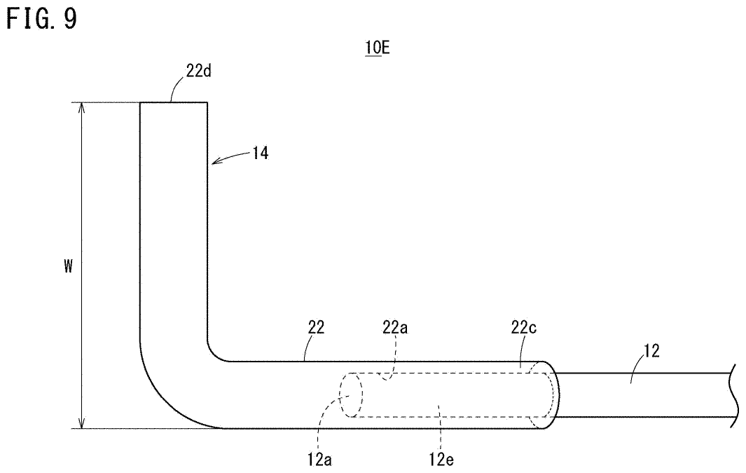

As illustrated in FIG. 9, an embolus material 10E according to the present embodiment includes the filling body 12 and a rod-shaped member 22 attached to the filling body 12 in the vicinity of the end portion 12a of the filling body 12. The rod-shaped member 22 is made of a shape memory alloy, and is shaped in advance such that a distal end 22d side is bent in an L shape to extend in a manner such as shown in FIG. 9. A proximal portion 22c side of the rod-shaped member 22 possesses a cylindrical shape, and an inner diameter of a recessed portion or blind hole 22a in the rod-shaped member 22 is substantially the same as the outer diameter of the filling body 12.

The distal side 12e of the filling body 12 is inserted into or positioned in the recessed portion 22a, and thus the filling body 12 is coupled to the rod-shaped member 22. The rod-shaped member 22 is initially provided in a state of being linearly stretched or straightened, but is deformed into an L shape as illustrated to form the shaped portion 14 when being positioned in the aortic aneurysm 92 and coming into contact with the body temperature. A shape into which the rod-shaped member 22 is shaped is not limited to the L shape, but may be various shapes such as a ring shape, a coil shape, and a spiral shape. The shaped portion 14 is formed to spread out in the range W wider than the inner diameter D of the branch blood vessel 94 connected to the aortic aneurysm 92. FIG. 9 illustrates one end of the embolus material 10E, it being understood that the opposite end of the embolus material 10E is similarly configured.

As mentioned above, the embolus material 10E of the present embodiment can achieve the same effect as that achieved by the embolus material 10 of the first embodiment.

### Seventh Embodiment

In the present embodiment, various modification examples of the shaped portions 14, 14B, and 14C of the respective embolus materials 10 to 10E described with reference to FIGS. 1 to 9 will be described.

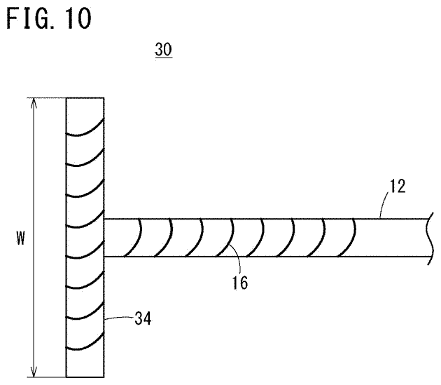

In an embolus material 30 according to a first modification example, as illustrated in FIG. 10, a shaped portion 34 is formed in a plane substantially perpendicular to an axial direction of the filling body 12. In other words, the shaped portion 34 is a two-dimensional structure, and has a T shape as illustrated when viewed from the side. The shaped portion 34 is shaped by the thin wire 16 that is wound in a coil shape on the outer circumferential portion of the filling body 12 and is formed of a shape memory member. The shaped portion 34 may be formed of the filling body 18 (refer to FIG. 5A) in which the core material 20 is positioned, the filling body 12C (refer to FIG. 7C) shaped by the mold 110, or the rod-shaped member 22 (refer to FIG. 9) instead of the thin wire 16.

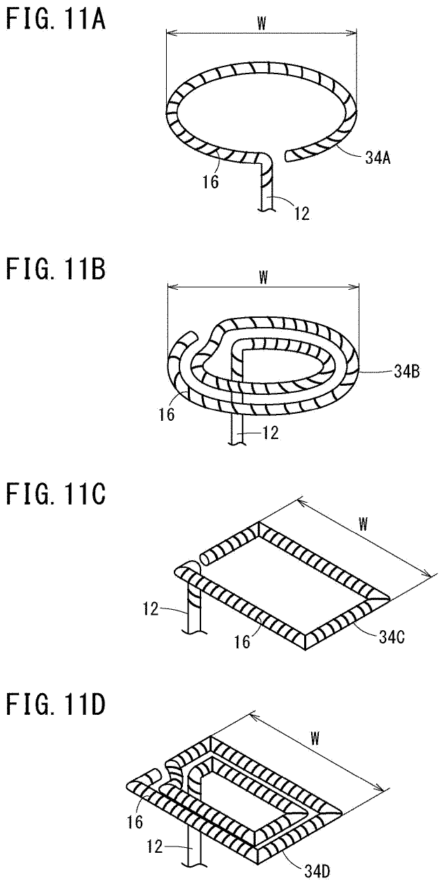

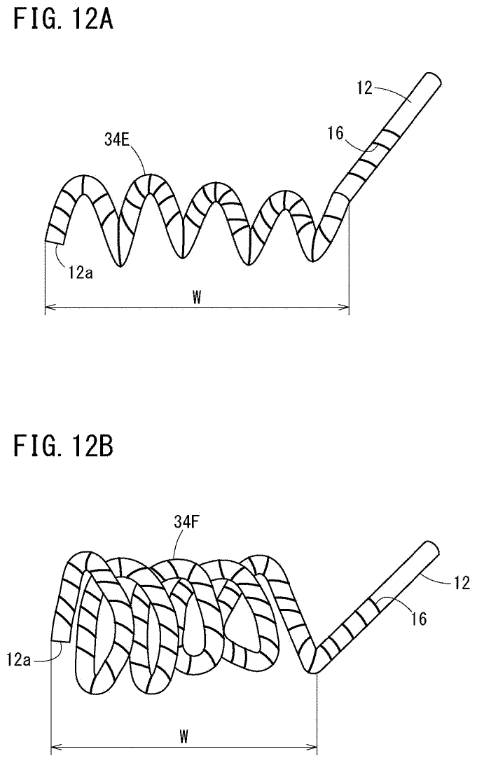

The shaped portion 34 of the embolus material 30 may have shapes as illustrated in FIGS. 11A to 12B. A shaped portion 34A according to a first configuration example of the present embodiment illustrated in FIG. 11A is shaped into a circular shape or an elliptical ring shape. A shaped portion 34B according to a second configuration example illustrated in FIG. 11B is shaped into a circular spiral shape or an elliptical spiral shape. The width W of the shaped portions 34A and 34B is larger than the inner diameter D of the branch blood vessel 94 connected to the aortic aneurysm 92.

A shaped portion 34C according to a third configuration example illustrated in FIG. 11C is shaped into a rectangular ring shape. A shaped portion 34D according to a fourth configuration example illustrated in FIG. 11D is shaped into a rectangular spiral shape. The width W of the shaped portions 34C and 34D in the long side direction is larger than the inner diameter D of the branch blood vessel 94 connected to the aortic aneurysm 92.

A shaped portion 34E according to a fifth configuration example illustrated in FIG. 12A is shaped into a corrugated shape. A shaped portion 34F according to a sixth configuration example illustrated in FIG. 12B is shaped to be entangled in the same plane. The width W of the shaped portion 34E and 34F is larger than the inner diameter D of the branch blood vessel 94.

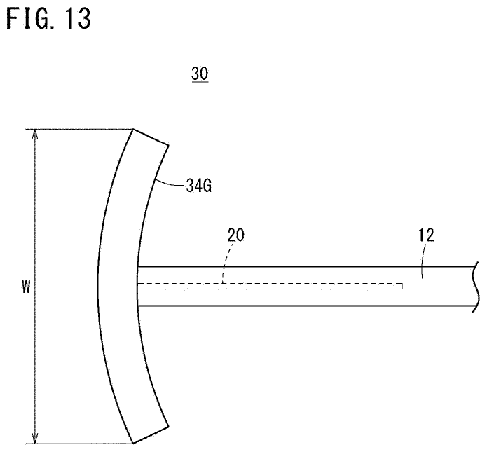

A shaped portion 34G according to a seventh configuration example illustrated in FIG. 13 is shaped to be curved like a bow when viewed from the side. The shaped portion 34G is shaped by the core material 20. A planar shape of the shaped portion 34G may be the same as any one of the shaped portions 34A to 34F illustrated in FIGS. 11A to 12B.

The same effect as the effect achieved by the embolus material 10 of the first embodiment can also be achieved by using the first to seventh configuration examples of the present embodiment.

### Eighth Embodiment

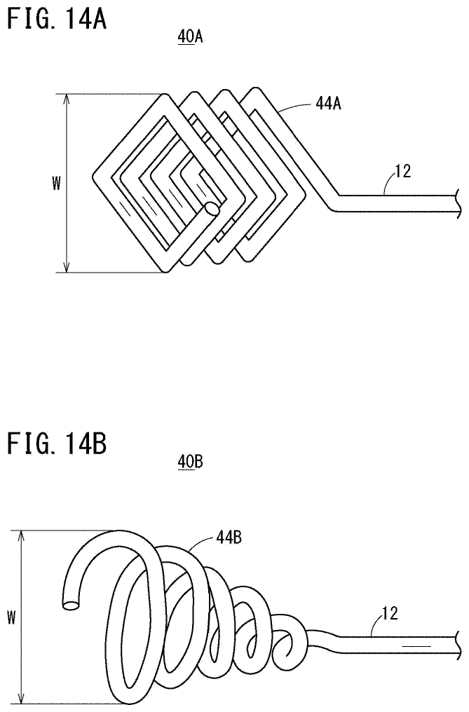

As illustrated in FIG. 14A, an embolus material 40A according to the present embodiment has a shaped portion 44A that is shaped into a rectangular coil shape at a terminal portion of the filling body 12. The shaped portion 44A is formed by a shape memory alloy inserted into the filling body 12. The width W of the shaped portion 44A of the present embodiment is larger than the inner diameter D of the branch blood vessel 94, and thus it is possible to prevent the embolus material 40A from entering the branch blood vessel 94.

As illustrated in FIG. 14B, an embolus material 40B according to a modification example of the present embodiment has a shaped portion 44B that is wound in a tornado shape (conically-shaped helix) at a terminal portion of the filling body 12. The shaped portion 44B is formed by a shape memory alloy filled into the filling body 12. The width W of the shaped portion 44B of the present modification example is larger than the inner diameter D of the branch blood vessel 94 connected to the aortic aneurysm 92, and so it is possible to prevent the embolus material 40B from flowing out to the branch blood vessel 94. Since the terminal end of the embolus material 40B is shaped, the risk of the distal end of the embolus material 40B sticking to the aortic aneurysm 92 and puncturing the aortic aneurysm can be reduced.

### Ninth Embodiment

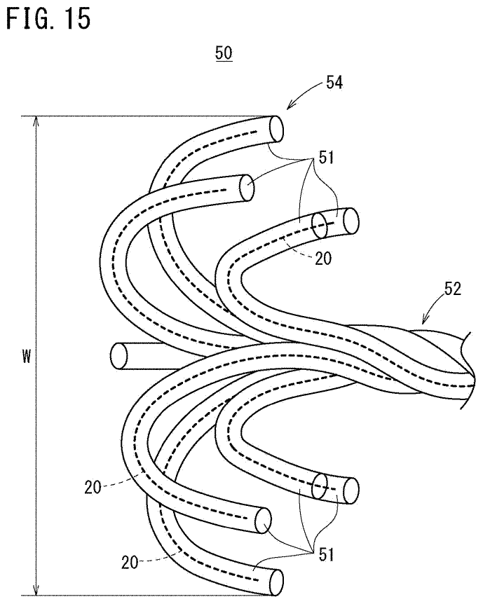

In an embolus material 50 according to the present embodiment, as illustrated in FIG. 15, a filling body 52 is formed by twisting a plurality of filaments 51 made of a hydrogel. A diameter (thickness) of the filling body 52 may be, for example, about 1 mm. The core material 20 shaped in advance into a U shape is inserted into or positioned in each filament 51 in the vicinity of a distal end of each filament 51 forming the filling body 52.

In a case where the embolus material 50 is provided in the aortic aneurysm 92 via the catheter 100, the core material 20 is deformed into the pre-shaped shape due to a body temperature. Consequently, a distal portion of the embolus material 50 is unraveled and the respective filaments 51 are branched, and the distal ends of the filaments 51 spread out to warp toward the proximal sides thereof. As a result, as illustrated, a shaped portion 54 of the present embodiment forms a structure in which the plurality of filaments 51 are developed in an umbrella shape. The shaped portion 54 is shaped such that the width W thereof is wider than the inner diameter D of the branch blood vessel 94.

For the shaped portion 54, instead of the filament 51 into which the core material 20 is built, the filament 51 on which the thin wire 16 is wound may be used, and a filament shaped in advance by the mold 110 may be used.

As mentioned above, the embolus material 50 of the present embodiment can also achieve the same effect as that achieved by the embolus material 10 of the first embodiment.

### Tenth Embodiment

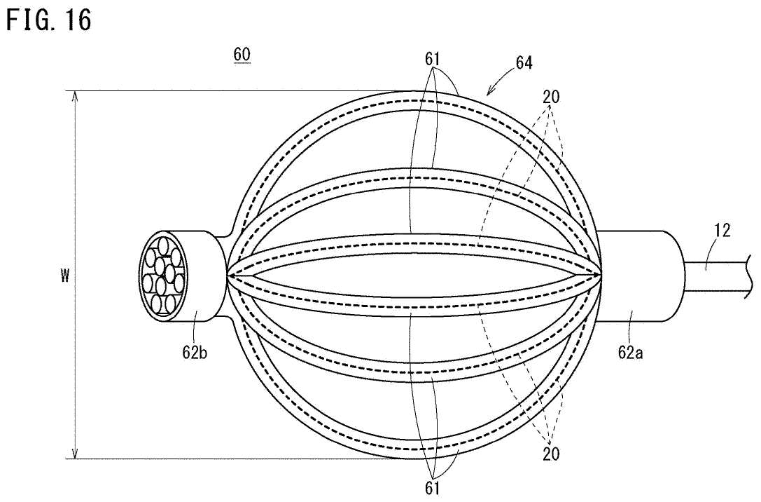

As illustrated in FIG. 16, an embolus material 60 according to the present embodiment includes bundled portions 62a and 62b in which a plurality of filaments 61 made of a hydrogel are bundled as a shaped portion 64. The filaments 61 are bundled at the bundled portion 62a on the proximal side and the bundled portion 62b on the distal side, and spread outwards in a radial direction between the two bundled portions 62a, 62b.

The core material 20 that is shaped in advance into a C shape to spread outwards in the radial direction is inserted into each filament 61. The core material 20 is made of a shape memory alloy, and is deformed into the pre-shaped shape when coming into contact with a body temperature. The filaments 61 including the core materials 20 are shaped into a linearly bundled shape, and the bundled portion 62a on the proximal side is coupled to the filling body 12.

When the embolus material 60 is provided in the aortic aneurysm 92 via the catheter 100, the respective filaments 61 forming the shaped portion 64 are deformed into a C shape due to a body temperature to form an illustrated ball-like structure. The width W of the shaped portion 64 of the present embodiment is larger than the inner diameter D of the branch blood vessel 94 connected to the aortic aneurysm 92. For the shaped portion 64, instead of the filament 61 into which the core material 20 is built, the filament 61 on which the thin wire 16 is wound may be used, and a filament shaped in advance by the mold 110 may be used.

As mentioned above, the embolus material 60 of the present embodiment can also achieve the same effect as that achieved by the embolus material 10 of the first embodiment.

### Eleventh Embodiment

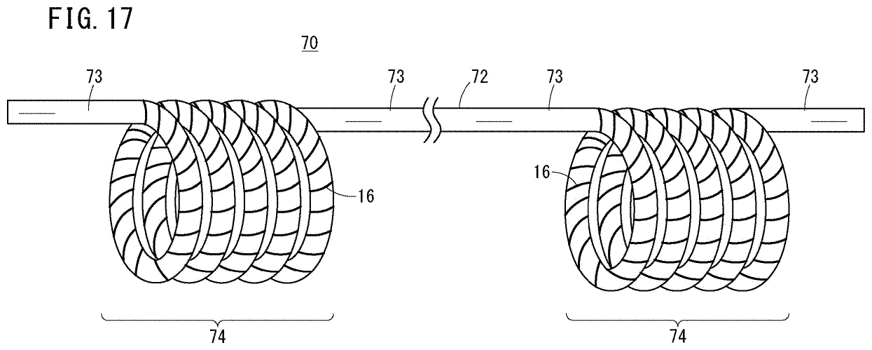

In an embolus material 70 according to the present embodiment, as illustrated in FIG. 17, a shaped portion 74 is provided in a part of a filling body 72, and a terminal end portion of the shaped portion 74 is a linear portion 73 that is not shaped. An example is illustrated in which two shaped portions 74 are provided in the illustrated filling body 72, but, the present embodiment is not limited to this illustrated example, and the number of shaped portions 74 may be one. The number of shaped portions 74 of the filling body 72 may be three or more. An embolus material may be used in which the shaped portion 74 is provided at one terminal end, and the linear portion 73 is provided at the other terminal portion.

A diameter of the filling body 72 is preferably smaller than the inner diameter D of the branch blood vessel 94. Consequently, the linear portion 73 easily enters the branch blood vessel 94. A shape of the shaped portion 74 in the embolus material 70 is not limited to a coil shape, and may have various shapes as illustrated in FIGS. 5A to 16. In the filling body 72 having the above-described configuration, the linear portion 73 at the terminal end thereof easily enters the branch blood vessel 94. The linear portion 73 of the filling body 72 is swollen to a diameter of 2 to 4 times in the branch blood vessel 94, and thus an effect that the linear portion 73 directly embolizes the branch blood vessel 94 can be expected.

### Twelfth Embodiment

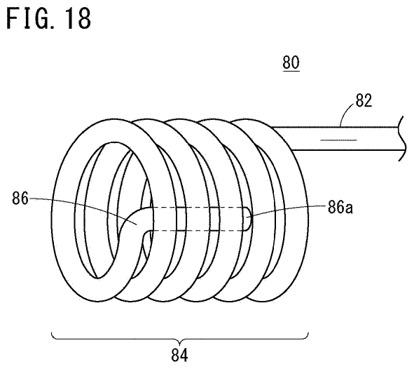

As illustrated in FIG. 18, an embolus material 80 according to the present embodiment has a spiral shaped portion 84 at a terminal portion of a filling body 82. A terminal portion 86 of the shaped portion 84 is shaped so that it is bent inward of the shaped portion 84, and a tip part 86a of the terminal portion 86 is disposed inside the shaped portion 84.

For the embolus material 80, for example, a raw material solution in which an ethylenically unsaturated monomer, which is a raw material for a hydrogel, a crosslinking agent, and a polymerization initiator are dissolved in a solvent is put in a tube. As this tube, for example, a tube having a predetermined diameter produced from HYTREL® (registered trademark) manufactured by DuPont may be used. Such a tube can be dissolved in a predetermined solvent, and the embolus material 80 can be easily taken out from the tube.

Next, the tube of a portion corresponding to the shaped portion 84 of the tube filled with the raw material solution is spirally wound around a hollow pipe-shaped mold. In this case, a terminal portion of the tube is placed inside a spirally wound portion of the mold that is folded back into the hollow portion.

Thereafter, the tube wound around the mold is heated to polymerize the inside solution, and thus a hydrogel is formed to form the embolus material 80. After the hydrogel is formed, the tube is removed with a solvent, the embolus material 80 is washed and is then stretched linearly and dried, and thus the embolus material 80 in a dried and contracted state is obtained.

The embolus material 80 and the manufacturing method thereof according to the present embodiment are as described above, and an operation of the embolus material 80 will be described below.

An embolus material formed by polymerizing a hydrogel in a mold or a tube tends to be softer than an embolus material shaped by a shape memory alloy or the like. When an end part of a shaped portion strays into a branch blood vessel, such an embolus material may be sucked into the branch blood vessel from a terminal side all at once, and thus the entire embolus material may stray into the branch blood vessel.

In contrast, according to the embolus material 80 of the present embodiment, the terminal portion 86 of the embolus material 80 is disposed inside the spiral shaped portion 84. Thus, since the terminal portion 86 does not stray into the branch blood vessel 94, the embolus material 80 can prevent the shaped portion 84 from being sucked into the branch blood vessel 94 from the terminal end. Since the terminal end of the embolus material 80 is shaped, the risk of the distal end of the embolus material 80 sticking to the aortic aneurysm 92 and puncturing the aortic aneurysm can be reduced.

The detailed description above describes embodiments of an embolus material and operational method representing examples of the inventive embolus material and method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An embolus material configured to be filled into an aortic aneurysm connected to a branch blood vessel via a catheter to inhibit inflow of blood into the aortic aneurysm from the branch blood vessel or outflow of the blood from an inside of the aortic aneurysm to the branch blood vessel, the embolus material comprising:
an elongated filling body that is sized smaller than an inner diameter of the catheter so that the elongated filling body is positionable in the catheter;
a shaped portion provided at least at a part of the elongated filling body, the shaped portion being configured to bend in a bent state or curve in a curved state after coming out of the catheter;
the shaped portion being configured to spread out, in the bent or curved state, wider than an inner diameter of the branch blood vessel after coming out of the catheter.

2. The embolus material according to claim 1, wherein the elongated filling body is made of a hydrogel that swells through contact with blood.

3. The embolus material according to claim 1, wherein the shaped portion includes a member made of shape memory material wound in a coil shape on an outer circumference of the filling body.

4. The embolus material according to claim 1, wherein the shaped portion includes a member made of shape memory material that is buried inside the elongated filling body so that the elongated filling body surrounds the member made of the shape memory material.

5. The embolus material according to claim 1, wherein the elongated filling body includes opposite ends, the shaped portion being provided at both ends of the filling body.

6. The embolus material according to claim 1, wherein the shaped portion extends over an entirety of the elongated filling body.

7. The embolus material according to claim 5, wherein the shaped portion is shaped so as to, after coming out of the catheter, spread out in the range wider than the inner diameter of the branch blood vessel in a width direction of the filling body by bending of the shaped portion with respect to the elongated filling body.

8. The embolus material according to claim 5, wherein the shaped portion is wound so that, after coming out of the catheter, the shaped portion is a wound shaped portion possessing a circular or polygonal ring shape having a diameter larger than the inner diameter of the branch blood vessel.

9. The embolus material according to claim 5, wherein the shaped portion, after coming out of the catheter, is umbrella-shaped having a diameter larger than the inner diameter of the branch blood vessel by virtue of a terminal end of the filling body being branched into a plurality of filaments that are turned back to a proximal side of the elongated filling body.

10. The embolus material according to claim 5, wherein the shaped portion after coming out of the catheter being ball-shaped at a terminal end of the elongated filling body and having an outer diameter larger than the inner diameter of the branch blood vessel.

11. The embolus material according to claim 1, wherein the shaped portion is configured to form a structure having a diameter of 3 mm or more after coming out of the catheter.

12. The embolus material according to claim 1, wherein the elongated filling body is made of a material that swells through contact with blood and has an outer diameter smaller than the inner diameter of the branch blood vessel in a state before being swollen such that a part of the filling body other than the shaped portion is able to enter and occlude the branch blood vessel.

13. The embolus material according to claim 12, wherein the elongated filling body includes a terminal portion that is linear and remains linear after coming out of the catheter.

14. A method comprising:
introducing a catheter into a body while an embolus material is positioned inside the catheter, the embolus material comprising an elongated filling body and a shaped portion provided at least at a part of the elongated filling body;
pushing the embolus material through an open distal end of the catheter while the catheter is in the body so that the embolus material comes out of the catheter and is introduced into an aortic aneurysm to inhibit inflow of blood into the aortic aneurysm from a branch blood vessel or outflow of the blood from an inside of the aortic aneurysm to the branch blood vessel; and
the shaped portion of the embolus material bending or curving after coming out of the catheter to spread out in a bent or curved state wider than an inner diameter of the branch blood vessel such that at least a portion of the embolus material remains in the aortic aneurysm without flowing out from the aortic aneurysm to a branch vessel connected to the aortic aneurysm.

15. The method according to claim 14, wherein the pushing of the embolus material through the open distal end of the catheter so that the embolus material comes out of the catheter causes the elongated filling body to swell to an increased size through contact with blood in the body.

16. The method according to claim 14, wherein the bending or curving of the shaped portion of the embolus material occurs by virtue of the shaped portion being a shape memory material on an outer circumference of the filling body.

17. The method according to claim 14, wherein the bending or curving of the shaped portion of the embolus material occurs by virtue of the shaped portion being shape memory material buried inside the elongated filling body so that the elongated filling body surrounds the member made of the shape memory material.

18. The method according to claim 14, wherein the shaped portion of the embolus material that bends or curves after coming out of the catheter is a first shaped portion located at one end of the elongated filling body, the elongated filling body also including a second shaped portion that is located at an opposite end of the elongated filling body and that also comes out of the catheter, the second shaped portion bending or curving after coming out of the catheter to spread out wider than the inner diameter of the branch vessel, the elongated filling body including an intermediate portion between the first and second shaped portions, the intermediate portion of the elongated filling body not bending or curving after coming out of the catheter.

19. The method according to claim 14, wherein the shaped portion extends beyond an end of the elongated filling body.

20. The method according to claim 14, wherein the elongated filling body includes a terminal portion that is linear before coming out of the catheter and is linear after coming out of the catheter.
